(19) United States
(12) Patent Application Publication
(10) Pub. No.: US 2025/0089752 A1
(43) Pub. Date: Mar. 20, 2025

(54) Microbiota of infants born by caesarean section

(71) Applicants: N.V. Nutricia, Zoetermeer (NL); Nutricia Early Life Nutrition (Shanghai) Co., Ltd., Shanghai (CN)

(72) Inventors: Harm Johannes Wopereis, Utrecht (NL); Guus Roeselers, Utrecht (NL); Mengjin Liu, Shanghai (CN); Yi Jin, Shanghai (CN)

(73) Assignees: N.V. Nutricia, Zoetermeer (NL); Nutricia Early Life Nutrition (Shanghai) Co., Ltd., Shanghai (CN)

(21) Appl. No.: 18/942,822

(22) Filed: Nov. 11, 2024

Related U.S. Application Data
(63) Continuation of application No. PCT/CN2022/092422, filed on May 12, 2022.

(51) Int. Cl. A23L 33/135; A23L 33/00; A23L 33/125

(52) U.S. Cl. CPC A23L 33/135; A23L 33/125; A23L 33/40

(57) ABSTRACT

The invention pertains to a composition for use in increasing the relative abundance of Bacteroides and Parabacteroides in the intestinal microbiota of an infant that was delivered by Caesarean section or of an infant that was exposed to antibiotics early in life (e.g. intrapartum antibiotic prophylaxis (IAP)), preferably an infant that was delivered by Caesarean section, wherein the composition comprises Bifidobacterium breve and non-digestible oligosaccharide(s).

### FIELD OF THE INVENTION

The invention relates to a nutritional composition for infants that have a compromised microbiota in the first weeks of life, such as infants born by Caesarean section and infants that are exposed to antibiotics during delivery or within the first week after birth, particularly infants born by Caesarean section, with the aim to improve the development of the intestinal microbiota.

### BACKGROUND OF THE INVENTION

The worldwide rate of infant deliveries via Caesarean section has increased over the last decade, making it the most common surgical procedure performed in women of childbearing age today. While the WHO recommends that Caesarean section deliveries be indicated in 10 to 15% of all deliveries, in many countries the rate of Caesarean section deliveries significantly exceeds this recommendation. In Europe alone 30% of the babies are delivered by Caesarean section.

Over the past years, the medical field has started to realize that a Caesarean section delivery introduces health risks. The obstetrician is advised to assess these long- and short-term health consequences to mother and infant, and weigh these risks with those associated with the surgery itself. A Caesarean born infant is at higher risk for an immune disorder like allergy, atopic dermatitis and asthma, and for infections such as upper respiratory tract infections. There are also indications for an increased risk of obesity and type-2 diabetes later in life, and associations with negative effects on cognition and behavioral changes have also been made. It is thought that the microbial dysbiosis early in life is playing a role; infants delivered via Caesarean section suffer from the consequences of a non-existent transmission of gut microbiota from the mother to the infant at birth.

EP1940250 has clarified that at birth there is a complete lack of any detectable amounts of Bifidobacterium species in the gut of C-section infants compared to the presence of significant amounts of at least three different Bifidobacterium species of the group of amongst others B. longum, B. breve, B. infantis, B. catenulatum, B. pseudocatenulatum, and B. bifidum observed in infants born by natural birth. While vaginally delivered infants acquire bacterial communities from their own mother's vaginal and gut microbiota, C-section infants acquire bacterial communities from the maternal skin and the surrounding birth environment. EP1940250 advocates the use of at least one Bifidobacterium species in combination with a non-digestible oligosaccharide, in order to normalize the gut microbiota in the infant. Promoting or stimulating healthy gut microbiota in infants delivered by Caesarean section is considered a therapeutic use, given that said infants suffer from bifidobacteria deficits at birth. Compared to the normal 10⁷-10⁹ cfu bifidobacteria counts, an infant born by Caesarean section typically exhibits hardly any bifidobacteria at birth. This can also be derived from Bennet et al. “Development of the faecal anaerobic microflora after caesarean section and treatment with antibiotics in newborn infants” Infection 15 (1987) page 332-336. Again with a focus on Bifidobacterium species, following up from EP1940250, WO 2019/115382 discloses a method for improving or stimulating the development of a healthy gut microbiota, by stimulating Bifidobacterium intra-species growth, in infants delivered via C-section, by administering a composition comprising Bifidobacterium breve and at least one non-digestible oligosaccharide, wherein the composition is administered within the first week after birth.

However, the difference in microbiota development between a vaginally born and Caesarean born infant is not only about the colonization by bifidobacteria. Also the development of Bacteroides is impaired. Appearance and increase of genera like Bacteroides and Parabacteroides are indicative of a normal development of the microbiota toward a more adult type of microbiota when the infant ages and starts weaning on complementary food. Up to one year of age, a lower or absent and less diverse population of Bacteroidetes (which comprises both the genus Parabacteroides and Bacteroides) were found in the infants born by Caesarean section (Jakobsson et al, Gut 2014, 63:559-66; Shao et al, Nature, 574:117-121). Differences in microbiota development can last up to 3 to 5 year of age.

WO 2019/215289 discloses nutritional compositions that are useful for promoting beneficial bacteria in the gastrointestinal tract of a Caesarean-section-delivered infant. The nutritional composition can include a prebiotic composition comprising human milk oligosaccharides, milk fat globule membrane (MFGM), and galacto-oligosaccharides (GOS) and/or polydextrose (PDX).

WO 2020/074569 discloses compositions comprising a bacterial strain of the genus Bacteroides, for use in a method of increasing the microbiota diversity and/or inducing stability of the microbiota of a subject including Caesarean section born infants. However, Bacteroides strains are not food-grade and therefore not suitable for Caesarean section born infants and strictly anaerobic, so expensive to incorporate in a product in an active form.

Therefore there is a need to provide a food-grade nutritional composition suitable for Caesarean born infants that not only address the microbiota dysbiosis related to bifidobacteria but also relating to the effect on intestinal Bacteroides and Parabacteroides.

### SUMMARY OF THE INVENTION

The faecal samples obtained in a clinical trial including both vaginally born infants and infants born by Caesarean section have been analyzed; the effect of intervention to the infants for at least 4 up to 6 months of age with a formula comprising Bifidobacterium breve and non-digestible oligosaccharides was assessed. The inventors surprisingly found that there was a catch-up in abundance of the genera Parabacteroides and Bacteroides towards the levels observed in vaginally-delivered and breast-fed infants in the group of Caesarean born infants that had received the formula, administered for up to 4 to 6 months of age. This effect was not observed in this group of Caesarean born infants that had received a formula that contained the non-digestible oligosaccharides but not B. breve. Also, in the breast-fed infants born by Caesarean section a Bacteroides imbalance in the gut microflora distribution was still detected, compared to the infants born by Caesarean section that received the formula of the invention.

On the other hand, in healthy, vaginally born infants no lack of Bacteroides was observed, and thus this improved effect of a formula including B. breve and non-digestible oligosaccharides over a formula with only non-digestible oligosaccharides was not observed.

The inventors thus found a way of restoring (Para) Bacteroides levels without having to resort to administering (Para) Bacteroides. Interestingly, the inventors found a programming effect; the effect of Bacteroides was mainly observed when the Caesarean born infant had reached an age of 12 months, at least 6 months long exceeding the time the formula comprising Bifidobacterium breve and the non-digestible oligosaccharides was administered. This is indicative for an effect of the formula going beyond a direct effect on the microbiota and that lasting effects on the microbiota development occurred, beyond the period of administration, said development being comparable to the microbiota development of healthy vaginally born infants. The above findings are believed to stand model also for infants whose mother received antibiotics during delivery, since the outcome of that likewise results in a lack of the appropriate gut microflora at the time of birth.

Based on these findings, the invention pertains to a composition for use in increasing the relative abundance of Bacteroides and/or Parabacteroides in the intestinal microbiota of an infant that was delivered by Caesarean section or of an infant that is exposed to antibiotics during delivery or within the first week after birth, wherein the composition comprises Bifidobacterium breve (B. breve) and non-digestible oligosaccharide(s). Worded differently, the invention also relates to the use of a composition in the manufacture of a product for use in increasing the relative abundance of Bacteroides and/or Parabacteroides in the intestinal microbiota of an infant that was delivered by Caesarean section or of an infant that is exposed to antibiotics during delivery or within the first week after birth, wherein the composition comprises Bifidobacterium breve and non-digestible oligosaccharide(s). B. breve and non-digestible oligosaccharide(s) are provided in therapeutically effective amounts. Also, the invention pertains to a (non-therapeutic) method for increasing the relative abundance of Bacteroides and/or Parabacteroides in the intestinal microbiota of an infant that was delivered by Caesarean section or of an infant that is exposed to antibiotics during delivery or within the first week after birth, wherein the infant is given a composition comprises Bifidobacterium breve and non-digestible oligosaccharide(s). Compared to the reference healthy infant that is vaginally delivered and who is not exposed to antibiotics early in life, the infant that is delivered by Caesarean section or infants exposed to antibiotics early in life is characterized by an impaired gut microflora distribution of Bifidobacterium, Parabacteroides and Bacteroides species, typically a lack of detectable amounts of Bifidobacterium, Parabacteroides and Bacteroides species. There is typically at least a factor 1000× difference when it comes to the difference in amounts of bacteria for each of Bifidobacterium, Parabacteroides and Bacteroides.

The invention is particularly directed to increasing the relative abundance of (the amount of) Bacteroides.

In a most preferred embodiment, the infant is delivered by Caesarean section.

### LIST OF FIGURES

FIGS. 1A and 1B report on Parabacteroides and Bacteroides levels in gut microbiome for the 4 groups (test group, VD; test group CS, control group, VD and control group CS). VD data are connected as solid lines, CS data are connected with dashed lines, with distinct lines for test (triangles), control (squares) and breastfed (circles) results. The figures show a significantly increased relative abundance in the Caesarean section born infant receiving the test formula. At the age of 4 months a significant difference (p=0.0006) and increase from baseline was observed (p=0.0047) in the level of Parabacteroides which was higher in the test group and became more comparable to the VD group. In the VD group on the other hand the effects of test vs control group on these microbiota genus was not significant. At 12 months a significant difference (p=0.0006) and increase from baseline was observed (p=0.0432) in the level of Bacteroides which was higher in the test group and became more comparable to the VD group. In the VD group, on the other hand, the effects of test vs control group on these microbiota genus was not significant.

### LIST OF PREFERRED EMBODIMENTS

### Detailed Description of the Invention

The invention pertains to a composition for use in increasing the relative abundance of bacteria of at least one genus selected from Bacteroides and Parabacteroides in the intestinal microbiota of an infant that was delivered by Caesarean section or of an infant that is exposed to antibiotics during delivery or within the first week after birth, wherein the composition comprises Bifidobacterium breve (B. breve) and non-digestible oligosaccharide(s). Worded differently, the invention also relates to the use of a composition in the manufacture of a product for use in increasing the relative abundance of bacteria of at least one genus selected from Bacteroides and Parabacteroides in the intestinal microbiota of an infant that was delivered by Caesarean section or of an infant that is exposed to antibiotics during delivery or within the first week after birth, wherein the composition comprises Bifidobacterium breve and non-digestible oligosaccharide(s). B. breve and non-digestible oligosaccharide(s) are provided in therapeutically effective amounts. Also, the invention pertains to a (non-therapeutic) method for increasing the relative abundance of bacteria of at least one genus selected from Bacteroides and Parabacteroides in the intestinal microbiota of an infant that was delivered by Caesarean section or of an infant that is exposed to antibiotics during delivery or within the first week after birth, wherein the infant is given a composition comprises Bifidobacterium breve and non-digestible oligosaccharide(s). Compared to the reference healthy infant that is vaginally delivered and who was not exposed to antibiotics early in life, the infant that is delivered by Caesarean section or an infant who is exposed to antibiotics early in life is characterized by an impaired gut microflora distribution of Bifidobacterium, Parabacteroides and Bacteroides species, typically a lack of detectable amounts of Bifidobacterium, Parabacteroides and Bacteroides species. There is typically at least a factor 1000× difference when it comes to the difference in numbers for each of Bifidobacterium, Parabacteroides and Bacteroides. The invention is particularly directed to increasing the relative abundance of bacteria of Bacteroides.

In a most preferred embodiment, the infant is delivered by Caesarean section. Caesarean section is a surgical procedure where an infant is delivered through an incision made in the mother's abdominal wall, and then through the wall of the uterus. A Caesarean section is typically performed when it is safer for the mother or the infant than a vaginal delivery, or in case the mother prefers to have a caesarean section rather than deliver her infant vaginally.

B. breve is provided in therapeutically effective amounts; it is provided in an amount sufficient for the composition to bring about the health effect strived for. The present composition preferably contains between 10³ and 10¹³ colony forming units (cfu) B. breve per gram dry weight of the present composition, preferably between 10⁴ and 10¹², more preferably between 10⁵ and 10¹¹. Preferably, the present composition contains between 10³ and 10¹³ colony forming units (cfu) bifidobacteria per g dry weight of the present composition, more preferably between 10⁴ and 10¹², most preferably between 10⁵ and 10¹¹. B. breve is preferably incorporated in the composition in an amount between 1*10⁶ and 1*10⁹ cfu per g powder. In terms of doses, the present composition preferably provides between 10³ and 10¹³ cfu, more preferably between 10⁴ and 10¹² cfu, most preferably between 10⁵ and 10¹¹ cfu B. breve per serving. Preferably, the present composition provides between 10³ and 10¹³ cfu, more preferably between 10⁴ and 10¹² cfu bifidobacteria per serving. B. breve is preferably incorporated in the composition to provide an amount between 1*10⁶ and 1*10⁹ cfu per serving. When the composition is administered to the infant in the form of or incorporated into infant or follow-on formula, there is typically 60-70 kcal/100 ml, and typically it is 13 g powder of infant or follow-on formula powder which is reconstituted per 100 ml. Based on those numbers, a suitable strain of B. breve to be used in the present invention is B. breve M-16V (Morinaga). The composition preferably provides between 10⁵ and 10¹⁴ colony forming units (cfu) Bifidobacterium breve per 100 ml ready-to-drink composition, more preferably between 10⁵ and 10¹³, even more preferably between 10⁶ and 10¹², most preferably between 1*10⁷ and 1*10¹⁰ cfu.

The inventors found that non-digestible oligosaccharide(s) is (are) also needed to achieve the effect i.e. increasing the relative abundance of Bacteroides and/or Parabacteroides, preferably increasing the relative abundance of Bacteroides in the intestinal microbiota of the infant. That is, it was found that the relative abundance of bacteria (bacterial cells, bacterial RNA) that belong to the genus Bacteroides and/or Parabacteroides, preferably the sum of all bacteria belonging to species of the genera Bacteroides) is increased. In the context of the invention, relative abundance is the way the abundance values are expressed from compositional data that are obtained with 16S rRNA-gene sequencing; most commonly expressed as the number of sequencing reads assigned to a specific bacterial taxon (e.g. Bacteroides) divided by the total number of sequencing reads for that sample. Without wishing to be tied down to any theory, the effect is attributed to the observation that it is only the combination of non-digestible oligosaccharides together with the B. breve which showed to be effective in stimulating bifidobacteria-abundance and lowering pH (compared to infant microbiota control; data not shown), the addition of B. breve is not sufficient. The non-digestible oligosaccharide(s) is (are) provided in therapeutically effective amounts; the non-digestible oligosaccharide(s) is (are) provided in an amount sufficient for the composition to bring about the health effect strived for. The abundance is preferably increased when comparing to the level of relative abundance in the intestinal microbiota of an infant born by Caesarean section that received a nutritional composition without Bifidobacterium breve. In a preferred embodiment, the abundance is increased to the level of relative abundance as observed in the intestinal microbiota of a vaginally born infant.

The term “non-digestible oligosaccharide” preferably refers to prebiotic oligosaccharides that beneficially affects the host by selectively stimulating the growth and/or activity of one or a limited number of bacterial species in the colon. Health effects of prebiotics, also in combination with probiotics, are described already in Collins, Am. J. Clin. Nutr. 1999, 69 (suppl.), 10525-10575. In the context of the present invention, the term “prebiotic oligosaccharide” refers to one or more non-digestible oligosaccharides. Advantageously, the non-digestible oligosaccharide is water-soluble (according to the method disclosed in L. Prosky et al, J. Assoc. Anal. Chem 71:1017-1023, 1988). Non-digestible oligosaccharides are not digested in the intestine by the action of digestive enzymes present in the upper digestive tract (small intestine and stomach) of the infant but instead are fermented by the intestinal microbiota of said infant, thus conferring benefits upon the host wellbeing and health. Preferably the growth of at least bifidobacteria and preferably also lactobacilli is stimulated. Preferably the non-digestible oligosaccharide(s) is (are) selected for at least stimulating the B. breve strain that is incorporated in the composition.

In a preferred embodiment, the non-digestible oligosaccharide is at least one selected from the group consisting of fructo-oligosaccharide, non-digestible dextrin, galacto-oligosaccharide, xylo-oligosaccharide, arabino-oligosaccharide, arabinogalacto-oligosaccharide, glucomanno-oligosaccharide, glucomanno-oligosaccharide, galactomanno-oligosaccharide, mannan-oligosaccharide, chito-oligosaccharide, uronic acid oligosaccharide, sialyloligosaccharide and fuco-oligosaccharide. Advantageously, the present composition preferably comprises non-digestible oligosaccharide(s) with an average degree of polymerization (DP) of 2 to 250, more preferably 2 to 100. Suitable non-digestible oligosaccharides are selected from the group consisting of fructo-oligosaccharide, non-digestible dextrin, galacto-oligosaccharide, xylo-oligosaccharide, arabino-oligosaccharide, arabinogalactooligosaccharide, gluco-oligosaccharide, glucomannooligo-saccharide, galactomanno-oligosaccharide, mannanoligo-saccharide, chito-oligosaccharide, uronic acid oligosaccharide, sialyl-oligosaccharide and fuco-oligosaccharide including 2′-fucosyllactose.

The present non-digestible oligosaccharide is preferably selected from the group consisting of galacto-oligosaccharides and fructo-oligosaccharides (including fructo-polysaccharides such as inulin) and 2′-fucosyllactose. Preferably at least 50 wt % of the present non-digestible oligosaccharides have an average degree of polymerization of 2 to 60. In one embodiment, the composition comprises galacto-oligosaccharides, in particular beta-galacto-oligosaccharides, more in particular trans-galacto-oligosaccharides. The galacto-oligosaccharides preferably comprise saccharides with an average DP of 2 to 10. (Trans)galactooligosaccharide is for example available under the trade name Vivinal®GOS (Borculo Domo Ingredients, Zwolle, Netherlands), Bimuno (Clasado), Cup-oligo (Nissin Sugar) and Oligomate55 (Yakult). Fructooligosaccharides may be inulin hydrolysate products having an average DP within the aforementioned (sub-) ranges; such FOS products are for instance commercially available as Raftilose P95 (Orafti) or Beneo® P95. Long-chain fructooligosaccharide is preferred, preferably having an average DP between 10 and 60, more preferably between 15 and 50. A particular type of long-chain fructo-oligosaccharides is inulin, such as Raftilin HP.

In a preferred embodiment, the composition comprises at least galacto-oligosaccharides, fructo-oligosaccharides and 2′-fucosyllactose, preferably in a weight ratio of 5:1:0.3-20:1:0.6, more preferably 7:1:0.3-15:1:0.6. In a particular preferred embodiment the present composition comprises at least galacto-oligosaccharides and fructo-oligosaccharides, preferably in a weight ratio of 5:1-20:1, more preferably 7:1-15:1, even more preferably 8:1-10:1, most suitably about 9:1.

The present composition suitably comprises 0.05 to 20 wt % of said non-digestible oligosaccharide(s), more suitably 0.5 to 15 wt %, even more suitably 1 to 10 wt %, most suitably 2 to 10 wt %, based on dry weight of the present composition. In a preferred embodiment, the composition suitably comprises 0.05 to 20 wt % of a mixture of galacto-oligosaccharides and fructo-oligosaccharides, more suitably 0.5 to 15 wt %, even more suitably 1 to 10 wt %, most suitably 2 to 10 wt %, based on dry weight of the present composition.

The present composition preferably provides 0.6-1 g, more preferably 0.7-0.9 g non-digestible oligosaccharide(s) per 100 ml of the product administered to the infant. The present composition preferably provides 0.12-0.2 g, more preferably 0.14-0.18 g non-digestible oligosaccharide(s) per 100 kcal of the product administered to the infant.

The composition according to the invention is typically suitable for enteral administration to the infant. The composition may be provided in any form known in the art to be suitable for such administration, such as in solid form, in semi-solid form or in liquid form. Preferably, the composition is a nutritional composition or a nutritional supplement. The composition may be referred to as a nutritional composition, preferably a nutritional composition for providing nutrition to infants, in particular infants delivered via Caesarean section.

In one embodiment, the composition is in the form of a powder supplement, which can be reconstituted with a liquid (typically water) to obtain a liquid composition. The composition may be provided in the form of a powder comprised in a single-use sachet, stick or stickpack, the powder providing—upon reconstitution—a serving. Alternatively, the composition is in the form of a suppository, pill or tablet, wherein the suppository, pill or tablet weighs between 0.1-10 gram per serving, more preferably between 0.2-5 gram per serving and most preferably 0.5-2.5 gram per serving.

In a preferred embodiment, the composition is administered as part of an infant formula powder or follow-on formula powder, to be reconstituted with a liquid (typically water) to obtain the corresponding infant formula or follow-on formula. The term ‘infant formula’ in the context of the invention and in the field is well-defined and controlled internationally and consistently by regulatory bodies. In particular, CODEX STAN 73—1981 “Standard For Infant Formula and Formulas For Special Medical Purposes Intended for Infants” is widely accepted. It recommends for nutritional value and formula composition, which require the prepared milk to contain per 100 ml not less than 60 kcal (250 kJ) and no more than 70 kcal (295 kJ) of energy. FDA and other regulatory bodies have set nutrient requirements in accordance therewith. There are similar guidelines for follow-on formulas.

The composition, preferably in reconstitutable or reconstituted form, is administered orally to, or intended to be administered orally to, the infant in need thereof and as defined herein, with an age of 0-6 months. The infants are preferably administered with the composition of the invention when 0-6 months of age only. With the term ‘only’ it is intended that the composition is preferably administered in said period exclusively. Since infant formula is predominantly the only nutrition that the infant is receiving, it is preferred that the composition is administered as part of the RTF infant formula.

The composition according to the invention, typically in the form of a formula, preferably comprises a lipid component, preferably a lipid component suitable for infant nutrition as known in the art. The lipid component of the present composition preferably provides 2.9 to 6.0 g, more preferably 4 to 6 g per 100 kcal of the composition. When in liquid form, the composition preferably comprises 2.1 to 6.5 g lipid per 100 ml, more preferably 3.0 to 4.0 g per 100 ml. Based on dry weight the present infant or follow-on formula preferably comprises 12.5 to 40 wt % lipid, more preferably 19 to 30 wt %.

The composition according to the invention may comprise further proteinaceous material. In the context of the present invention the additional “protein” or “proteinaceous material” or “protein equivalents” encompasses proteins, peptides, free amino acids and partially or extensively hydrolysed proteins.

In a preferred embodiment, the composition comprises hydrolysed cow's milk protein. In a preferred embodiment, the protein is a partial protein hydrolysate. These partially hydrolysed proteins have a decreased allergenicity. This approach has been demonstrated to be efficient in order to prevent sensitization by native proteins present in the adapted formulae. Typically, the extent of hydrolysis of proteins is less than those of extensively hydrolysed proteins for babies already suffering from allergy. The partial protein hydrolysate is preferably milk protein hydrolysate, more preferably whey protein hydrolysate. It is preferred that the degree of hydrolysis is between 5 and 25%. As Caesarean section born infants have an enhanced risk of developing allergy, linked to the altered microbiota development, even with the improved gut microbiota development achieved by the present invention, these C-section born infants could further benefit from the presence of partially hydrolysed proteins in the composition, preferably in the form of an infant or follow-on formula.

The present composition preferably contains 4 to 25%, more preferably 5 to 20%, more preferably 7 to 16%, most preferably 7 to 12% protein, based on total calories. The present composition, when in liquid form, preferably contains 0.5 to 6.0 g, more preferably 0.8 to 3.0 g, even more preferably 1.0 to 2.5 g of protein per 100 ml. The present composition preferably comprises at least 7.0 wt %, more preferably at least 8.0 wt %, most preferably at least 9 or at least 10 wt % protein based on dry weight of the total composition. Preferably, the present composition comprises at most 40 wt %, more preferably at most 15 wt %, preferably at most 20 wt % of protein based on dry weight of the total composition.

The composition may comprise digestible carbohydrate(s). Typically, digestible carbohydrates that are known in the art to be suitable for use in infant nutritional compositions are used, for example selected from digestible polysaccharides (e.g. starch, maltodextrin), digestible monosaccharides (e.g. glucose, fructose), and digestible disaccharides (e.g. lactose, sucrose). Particularly suitable is lactose and/or maltodextrin. The digestible carbohydrate component preferably comprises at least 60 wt % lactose based on total digestible carbohydrate, more preferably at least 75 wt %, even more preferably at least 90 wt % lactose based on total digestible carbohydrate.

The administration is preferably started within 2 months from birth, more preferably within the first month after birth, and preferably continued for at least 4 months after birth. The composition is preferably administered on a daily basis i.e. at least once a day. The administration is preferably discontinued at the latest after 6 months of age. The effect on the relative abundance of Bacteroides and Parabacteroides is observed later in life, when the infant has reached an age of 8 months or older, preferably 12 months or older. With the term ‘later in life’ it is understood an age exceeding the age at which the diet is taken, preferably exceeding said age with at least four months. The administration of the composition of the invention preferably results in a relative abundance of bacteria of Bacteroides and/or Parabacteroides in the intestinal microbiota of the infant to an extent which is comparable to that of the reference healthy infant (that is vaginally delivered and that was not exposed to antibiotics early in life) at least 6 months after the administration is discontinued. With the term ‘comparable’ in the context of the present invention it is understood that the abundance of Bacteroides and/or Parabacteroides for the targeted infants is less than 10× smaller than observed for the healthy vaginally-delivered counterpart.

it is preferred that the administration with the composition of the invention starts within 2 months from birth, preferably within 1 month from birth.

Given the similarities in gut microflora with the studied C-section delivered infants at birth, the inventors believe that the intervention also works advantageously for vaginally delivered infants who have been exposed to antibiotics early in life. In the context of the invention, the term ‘early in life’ preferably means within one week from birth, more preferably within 24 hours from birth, and is not limited to postnatal antibiotics exposure, but also includes exposure during delivery. In a preferred embodiment, the infant is an infant whose mother received antibiotics during delivery (Intrapartum Antibiotic Prophylaxis/IAP). In one embodiment, the infant born with a lack of Bacteroides and Parabacteroides species at birth is an infant who has been exposed to antibiotics early in life, preferably within one week from birth, more preferably within 24 hours from birth, most preferably an infant whose mother received IAP. It is known that within the group of C-section infants, an additional effect of postnatal antibiotics exposure or of intrapartum antibiotic prophylaxis (IAP) with or without clamping of the umbilical cord cannot be made. In other words the vertical transfer of microbes from mother-to-infant is already that much compromised by CS-delivery that postnatal antibiotics or IAP does not add a measurable effect to it.

### Example

### Clinical Trial—Setup

In a clinical trial 285 healthy, term infants were included, of which 112 were randomized to receive an experimental formula, 112 were randomized to receive the control formula and 60 healthy non-randomized infants formed the breastfed reference group.

TABLE 1
Birth mode characteristics of All Subjects Randomized and
breastfed subjects by study group of whom at least one
stool sample was collected within visit window and with
at least one stool parameter with a non-missing result.

| Mode of | | Test | Control | Total | Breastfed |
|---|---|---|---|---|---|
| delivery | Statistics | [N = 112] | [N = 109] | [N = 221] | [N = 58] |
| Vaginal | n (%) | 41 | 59 | 100 | 32 |
| | | (36.6%) | (54.1%) | (45.2%) | (55.2%) |
| Caesarean | n (%) | 71 | 50 | 121 | 26 |
| section | | (63.4%) | (45.9%) | (54.8%) | (44.8%) |

The infants were enrolled before they were 44 days old, and they were fully formula-fed by the time of randomization. In the experimental group 63.4% of the infants was born via Caesarean section. In the test group this was 45.9%. A faecal sample and saliva sample was taken at the start of the intervention (visit 1). The control or experimental formula was administered until the infant was 17 weeks, 4 months, of age at which time a faecal sample was taken. The breast fed reference group was at least fully breast fed up to 17 weeks of age.

After the infant was 17 weeks of age the parents had the option to continue with the formula for another 13 weeks until the infant reached an age of 26 weeks, 6 months. After that any feeding was allowed. When the infant was 52 weeks, 1 year, of age again a faecal sample was taken. Faecal samples were collected by the parents or caregivers on the day of visit or within 3 days prior to the visit to the clinic. Collected samples were stored in household freezers, transported with ice-packs to the clinic site and stored in ultra-low temperature freezers (−80° C.) at the clinic site. Transport to the laboratory was done on dry-ice and stored at −80° C. until further downstream processing for analysis.

The experimental formula was a formula having as a non-digestible oligosaccharide mixture galacto-oligosaccharides (source Vivinal GOS) and long chain fructo-oligosaccharides (lcFOS; source Raftilin HP) in a wt/wt ratio of 9:1 and in an amount of 0.8 g per 100 ml ready to drink formula. It contained Bifidobacterium breve M-16V (from Morinaga) in an amount of 3*10⁷ cfu/g powder (which is about 4*10⁸ cfu per 100 ml). As a source of protein partially hydrolysed whey protein from cow's milk was present. The control formula contained the same amount of GOS and lcFOS in the same ratio, but did not contain Bifidobacterium breve. As a protein source intact protein from cow's milk (whey protein and casein, wt/wt 6:4) was present. Further details of the clinical study, the formulas and the participant flow can be found in Wang et al, 2021, Nutrition 91-92:111472.

### Microbiota Analysis

Faecal samples were defrosted on ice, homogenized, and 0.2-0.3 gram was used to extract DNA using the QIAmp DNA Stool Mini Kit (Qiagen) according to the manufacturer's protocol with the addition of two bead-beating steps as described in Mischke et al. 2018. Diabetes Obes Metab 20 (6): 1408-1418.

The microbiome composition was determined on the DNA extracts of faecal samples collected at visit 1, visit 5 (17 weeks of age) and visit 8 (12 months of age) by 16S rRNA-gene sequencing as described by Wopereis et al. 2019. Clin Transl Allergy 9(1): 27. In short, The V3-V4 region of the 16S rRNA gene was PCR-amplified and sequenced on the MiSeq platform (Illumina) using the 2×300 bp paired-end protocol. Sequencing was performed by Beijing Genomics Institute (Shenzhen, China). The read pairs were demultiplexed and trimmed (q>20) before being merged using PEAR (Zhang et al. 2014. Bioinformatics 30 (5): 614-620.) Merged reads with q>25 over a window of 15 bases, no ambiguous bases and a minimal length of 300 were retained. These were dereplicated and counted using mothur (Schloss et al. 2009. Appl Environ Microbiol 75 (23): 7537-7541.) and reads with a low abundance (less than 2 reads over all samples) were discarded. Chimeras were removed using VSEARCH (Rognes et al. 2016. PeerJ 4: e2584.), using the RDP gold database (Haas et al. 2011. Genome Res 21 (3): 494-504.) as reference. Reads which contained PhiX or adapters as defined in Deblur (Amir et al. 2017. mSystems 2(2): e00191-00116.) as well as reads with a low relative abundance up to 0.0005% were eliminated. Taxonomic assignment was performed using the RDP classifier (Wang et al. 2007. Appl Environ Microbiol 73 (16): 5261-5267.) against the SILVA138 database (Pruesse et al. 2007. Nucleic Acids Res 35 (21): 7188-7196.). Reads with eukaryotic assignments, as well as reads assigned to genera with less than 50 reads in less than 5 samples were excluded from further downstream analysis. Unsupervised oligotyping of Bifidobacterium affiliated reads was applied to discriminate bifidobacterial species using the Minimum Entropy Decomposition (MED) algorithm (Eren et al. 2015. ISME J 9 (4): 968-979.). Alpha-diversity indexes were calculated using the phyloseq (McMurdie and Holmes 2013. PLOS One 8(4): e61217.) and vegan (Oksanen et al., 2020. https://CRAN.R-project.org/package=vegan) packages in R (https://www.R-project.org/). Statistical analysis was applied to all subjects randomized with fecal samples available and significance at 5%. Relative abundances of taxa were analysed by multivariate ordination methods using Canoco 5 (Ter Braak & Smilauer P. 2018: Canoco reference manual and user's guide: software for ordination, version 5.10. Microcomputer Power, Ithaca, USA, 536 pp.). Differential abundances of taxa across visits were analysed by either a negative binomial (NB) or zero-inflated negative binomial (ZINB) mixed models (depending on the magnitude of zero counts for each taxon tested) using the glmmTMB package in R (Brooks et al. 2017. The R Journal. 9(2). 378-400.). Alpha-diversity indexes (Observed and Shannon) were analysed with repeated measures mixed models using SAS® (SAS Enterprise Guide version 4.3 or higher) for Windows (SAS Institute Inc., Cary, NC).

### Results

At week 17 there was a significant change from baseline in increase in the observed Bifidobacterium oligotypes (species richness) in the test group when compared with the control group, for the infants born by Caesarean section (p=0.024). For the vaginally born infants such difference between control and test group was not observed. The Shannon diversity for Bifidobacteria oligotypes (species richness and species evenness) was at week 17 significantly higher in the Caesarean section born infant that had consumed the experimental formula (p=0.026) and became more comparable to the vaginally delivered formula-fed infants. In the vaginally born group the difference between the test and control group was not statistically significant. These numbers are summarized in Tables 2a and 2b (Bacteroides) and Tables 3a and 3b (Parabacteroides).

At genus level the gut microbiome was comparable at week 17 between test and control formula when looking at the total group of formula fed infants. However, the mode of birth had a large impact and an aberrant development of microbiota in Caesarean-section born infants was observed at week 17, which was marked by a reduced relative abundance of Parabacteroides and Bacteroides and an elevated abundance of potential pathogens such as Klebsiella when compared to vaginally-delivered infants. This is a known effect of the impact of Caesarean section delivery on microbiota development.

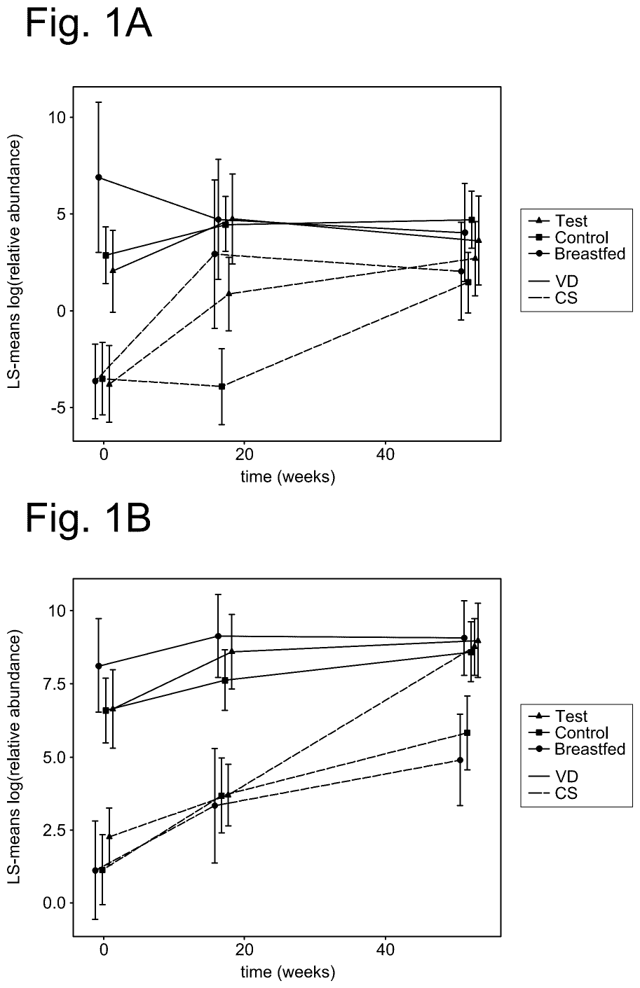

The development of Parabacteroides and Bacteroides in the gut microbiome in both control and intervention infant groups, for different modes of delivery, is plotted in FIGS. 1A and 1B, respectively. The breast-fed reference is also added.

When looking at the 4 groups (test group, VD; test group CS, control group, VD; and control group CS), a significantly increased relative abundance was observed in the Caesarean section born infant receiving the test formula. At the age of 4 months a significant difference (p=0.0006) and increase from baseline was observed (p=0.0047) in the level of Parabacteroides which was higher in the test group and became more comparable to the VD group. In the VD group on the other hand the effects of test vs control group on these microbiota genus was not significant. At 12 months a significant difference (p=0.0006) and increase from baseline was observed (p=0.0432) in the level of Bacteroides which was higher in the test group and became more comparable to the VD group. In the VD group, on the other hand, the effects of test vs control group on these microbiota genus was not significant.

1. A method comprising the step of:
administering a composition comprising Bifidobacterium breve and non-digestible oligosaccharide(s) to an infant, wherein the infant:
was delivered by Caesarean section,
was exposed to antibiotics during delivery,
was exposed to antibiotics within the first week after birth,
and combinations thereof,
wherein the administration of the composition increases the relative abundance of Bacteroides, and/or Parabacteroides, and combinations thereof in the intestinal microbiota of the infant.

2. The method according to claim 1, wherein the relative abundance is increased to a level of relative abundance as observed in the intestinal microbiota of a vaginally born infant.

3. The method according to claim 1, wherein the infant is an infant whose mother received antibiotics during delivery (i.e. intrapartum antibiotic prophylaxis (IAP)).

4. The method according to claim 1, wherein the increase in relative abundance is observed when comparing to the level of relative abundance in the intestinal microbiota of an infant born by Caesarean section that received a nutritional composition without Bifidobacterium breve.

5. The method according to claim 1, wherein the nutritional composition is administered to an infant between 0 and 6 months of age.

6. The method according to claim 1, wherein the effect on the relative abundance of Bacteroides is observed later in life when the infant has reached an age of 8 months or older.

7. The method according to claim 1, wherein administration to the infant is discontinued at the latest when the infant is 6 months of age, and wherein the effect on the relative abundance of Bacteroides is observed for at least 4 months after the administration is discontinued.

8. The method according to claim 1, wherein administration to the infant is discontinued at the latest when the infant is 6 months of age, and wherein the administration results in a relative abundance of species of Bacteroides in the intestinal microbiota of the infant to an extent which is comparable to that of the reference healthy vaginally delivered infant at least 6 months after the administration is discontinued.

9. The method according to claim 1, wherein the non-digestible oligosaccharide(s) are galacto-oligosaccharides, fructo-oligosaccharides, 2′-fucosyllactose and combinations thereof.

10. The method according to claim 1, wherein the level of Bifidobacterium breve is between 10³ and 10¹³ colony forming units (cfu) per gram dry weight of the present composition.

11. The method according to claim 1, wherein the composition comprises 0.05 to 20 wt % of non-digestible oligosaccharide(s) based on dry weight of the composition.

12. The method according to claim 1, wherein the nutritional composition is or is incorporated in an infant formula or follow-on formula.

13. The method according to claim 1, wherein the nutritional composition comprises partially hydrolysed protein.

14. (canceled)

15. (canceled)

16. The method according to claim 1, wherein the composition increases the relative abundance of Bacteroides.

17. The method according to claim 1, wherein the infant was delivered by Caesarean section.

18. The method according to claim 1, wherein the effect on relative abundance of Bacteroides is observed at an age exceeding the age at which the administered composition was discontinued.

19. The method according to claim 1, wherein the non-digestible oligosaccharide(s) are galacto-oligosaccharides and long-chain fructo-oligosaccharides.

20. The method according to claim 1, wherein the composition comprises 1 to 10 wt. % of non-digestible oligosaccharide(s), based on dry weight of the present composition.

21. The method according to claim 1, wherein the nutritional composition is an infant formula.

22. The method according to claim 1, wherein the nutritional composition comprises partially hydrolysed whey protein.
